(19) United States
(12) Patent Application Publication
(10) Pub. No.: US 2023/0338611 A1
(43) Pub. Date: Oct. 26, 2023

(54) BIOACTIVE IMPLANT FOR THE RESTORATION OF THE CONDUCTIVITY OF BIOELECTRIC STIMULI IN THE CARDIAC TISSUE

(71) Applicant: UNIVERSIDAD PONTIFICIA BOLIVARIANA, Medellín (CO)

(72) Inventors: John Jairo BUSTAMANTE OSORNO, Medellín (CO); Yuliet Marlinde MONTOYA OSORIO, Medellín (CO); Adriana María RESTREPO OSORIO, Medellín (CO)

(21) Appl. No.: 18/245,817

(22) PCT Filed: Sep. 17, 2021
(86) PCT No.: PCT/IB2021/058505

§ 371 Date: Mar. 17, 2023

(30) Foreign Application Priority Data
Sep. 19, 2020 (CO) NC2020/0011532

(51) Int. Cl. A61L 27/04; A61L 27/48; D01F 4/02; D01F 1/09; D01D 5/00

(52) U.S. Cl. CPC A61L 27/047; A61L 27/48; D01F 4/02; D01F 1/09; D01D 5/0038; D01D 5/0076; D01D 5/0069; A61L 2430/20; A61L 2400/12; A61L 2400/18; D10B 2509/00; D10B 2401/16; D10B 2211/22

(57) ABSTRACT

This innovation discloses a bioactive and biocompatible implant that comprises a fibrillar membrane of an electrospun polymeric matrix, where the matrix features a structural reinforcement with gold nanoparticles that allows the propagation of bioelectrical activity of the cardiac tissue, restoring the conductivity of the bioelectrical stimuli, given the electroconductive capacity provided by its components.

### FIELD OF THE INVENTION

The present invention is related to the technical field of tissue engineering, specifically implants for the bioconductivity of the cardiac tissue, providing a bioactive and biocompatible implant with electroconductive capacity, which allows the propagation of bioelectric activity, with structural and chemical characteristics that prompt the restoration of the conductivity of bioelectric stimuli in the myocardial tissue.

### BACKGROUND OF THE INVENTION

The heart walls are anatomical structures made up of both contractile elements and electrical propagation elements, configuring a mixed element featuring both biomechanical and bioelectric characteristics, which gives them a functional dimension of electromechanical coupling.

Faced with multiple structural changes derived from myocardium or endocardium pathologies, their bioelectric and mechanical behavior is modified, altering the functional activity, by either creating dyskinetic or akinetic areas, or provoking dysrhythmias. Thus such local lesions are to be overlapped in order to obtain a functional continuum to replicate the physiological model and restore the ideal behavior of the electromechanical coupling.

The mechanisms responsible for such dysrhythmias leading to impulse conduction disorders have motivated some groups to study cell interactions, developing different methods, including in vitro co-culture models, in order to establish processes involved in such pathologies and thus visualize an appropriate treatment. These studies have reported the effects of the propagation of the action potential, but little has been reported on possible local therapies for bioelectrical tissue block.

In the search for a biomaterial that emulates the functional behavior and provides a favorable biocompatible response with the cardiovascular system, different natural biopolymers, such as collagen, chitosan, keratin, among others, have been studied. These natural biopolymers have shown to have biocompatible characteristics, controllable biodegradation rate and adequate mechanical and structural properties. For all the above reasons, they are considered materials of interest for use in the field of cardiovascular tissue engineering.

However, such studies have focused mainly on solving congenic anatomic defects, valve repair, vascular lesions and tissue trauma.

Thus, in the state-of-the-art, there is no evidence of alternative therapeutic developments to the conventional use of pacemakers for conduction blocks. Consequently, although some works disclose therapeutic models with different implanted electrodes, they fail to divulge an effective mechanism to simulate the bioelectrical properties of a tissue implant, that is, a bioactive structure with electrical properties to perform properly in a specific biological environment and at the same time, is not harmful to the body.

In the field of invention patents, there are precedents such as document CN110859996A. This document discloses a cardiac patch that is made up of an elastic film that includes a biodegradable material, as well as a porous structure that comprises a biodegradable material, where the elastic membrane is located over the porous structure. The material to construct the cardiac patch comprises a mixture of polyprolactone, gelatin, polysebacic glyceric acid, and combinations thereof. Furthermore, this patch is made up of a porous structure with various biomimetic layers. However, document CN110859996A does not disclose the electrical conductive characteristics of the cardiac patch for problems originated in tissue blocks that affect the cardiac electrical conduction system.

On the other hand, document CN109847106A discloses a method to prepare conductive porous scaffolds for tissue engineering. Such method consists on preparing a solution of silk fibroin, albumin and polypyrrole, which is modulated with sodium chloride and overnight in refrigerator to create a porous structure. However, document CN109847106A does not disclose the electrophysiological functional assessments in cardiac cells and the disruption patterns of the cardiac bioelectrical stimulus.

Consequently, although the cited documents disclose methods to obtain biomaterials and patches in the field of cardiac tissue engineering, said documents do not disclose how to obtain an electrospun biomatrix with biofunctional capacity in a disruption model of bioelectric stimuli of cardiac cells. In addition, the state of the art has not proposed effective solutions to provide nanoreinforced biomatrixes with electroconductive capacity and a tridimensional fibrillary structure for applications in cardiac tissue engineering.

Under these conditions, the present invention solves the problems related to the procedures and the use of medical devices, such as, cardiac stimulators, pacemakers and resynchronizers, which bring with them complications associated to the implant, that may be related to healing disorders, bruising and infections. On the other hand, and in addition to this, the therapeutic devices present some disadvantages such as the battery discharge, and surrounding fibrosis associated with the implantation of the electrodes, requiring replacement procedures, which increases the number of interventions and the risk of complications. The present invention involves bioelectric connectivity of the cardiac tissue through an implant of a bioactive biomaterial of natural origin with electroconductive capacity as a response to the problems raised regarding the electrical conduction blocks in the cardiac tissues.

### BRIEF DESCRIPTION OF THE INVENTION

In a first object, the present invention corresponds to a bioactive implant or patch, that comprises at least one fibrillary membrane of an electrospun polymeric matrix, with a structural reinforcement of gold nanoparticles that, together, allow to increase the electroconductive properties of the biomatrix.

In a particular embodiment of the invention, the fibrillary membrane corresponds to a silk fibroin, reinforced with gold nanoparticles, obtained with the same protein. The silk fibroin (SF) is a natural protein that has been shown to have biocompatible characteristics and an adequate biodegradation capacity. The mechanical and electrical properties of the SF are used in the bioactive patch of the present invention through its configuration as a connection matrix in bioelectrical environments.

The bioactive implant or patch object of the present invention facilitates the biological, bioelectrical interaction and specific tissue repair, through the relationship of synthetic and natural materials with diverse properties present in its structure. The electroconductive polymeric matrix is reinforced with gold nanoparticles to intervene block patterns in the excito-conductive system, which was functionally evaluated in in vitro models of cardiac cell cultures.

The electroconductive polymeric matrix of the present invention was developed using the rotary sequential electrospinning technique. In the preferred modality of this invention, silk fibroin obtained from silk residues was used as natural biomaterial, reinforced with metallic nanoparticles such as gold, silver, platinum, palladium, which were obtained by green synthesis with the silk fibroin. Green synthesis is understood as the physical-chemical method that uses innocuous, non-toxic reagents that are ecological and biosafe, as reducing agents of protein origin, plant and fruit extracts, polysaccharides, among others.

In a second object, the present invention discloses a method to manufacture the bioactive implant or patch, which comprises the stages from the procurement of the silk fibroin material, its interaction with gold nanoparticles, and its final arrangement in a non-woven fibrillar structure obtained by electrospinning technique. Subsequently, the patch is completely permeated with cell adhesion proteins arranged over the matrix.

The previously described objects, as well as any additional object as may be applicable, will be exposed in detail and with the necessary sufficiency in the descriptive chapter below, that will constitute the basis of the claim chapter.

### BRIEF DESCRIPTION OF THE FIGURES

FIG. 1 illustrates the process for obtaining the silk fibroin from silk residues.

FIG. 2 illustrates the scheme of the interaction of neonatal cardiomyocytes with the electrospun biomatriz reinforced with gold nanoparticles, in a bioelectrical disruption pattern.

FIG. 3a illustrates the absorption spectra of the surface plasmon resonance and FIG. 3b shows micrographs of the gold nanoparticles.

FIG. 4a illustrates the fibrillar morphology of the biomatrix before and after removing the synthetic polymer, where the gold nanoparticles contained in the fibers are observed. FIG. 4b shows the absorption spectra of the biomatrix reinforced with gold nanoparticles.

FIG. 5 illustrates the FTIR spectra that reveals the chemical changes that conform the functional groups of the electrospun biomatrices.

FIGS. 6a, 6b and 6c illustrate the electrical behavior of the resistivity, conductivity and impedance of the electrospun biomatrix with and without gold nanoparticles post-treated with methanol.

FIGS. 7a, 7b, 7c y 7d illustrate the topography and the surface potential of the nanoreinforced biomatrix with and without methanol post-treatment. FIG. 7e shows the electrical potential behavior (mV) versus the position (μm) of the biomatrix reinforced with gold nanoparticles with and without post-treatment.

FIGS. 8a, 8b, 8c and 8d show the cellular structural changes of cardiomyocytes interacting with the biomatrix in bright field and immunofluorescence tests.

FIGS. 9a and 9b illustrate representative signals of normalized intensity of calcium fluorescence from cardiomyocytes with and without interaction with the biomatrix.

FIGS. 10a and 10b illustrate representative normalized intensity signals of cardiomyocyte membrane potential fluorescence with and without interaction of the biomatrix.

FIGS. 11a and 11b illustrate representative biosignals from neonatal cardiomyocytes with and without interaction with the biomatrix, obtained from microelectrode array matrices.

FIGS. 12a, 12b, 12c, 12d and 12e illustrate representative voltage maps of neonatal cardiomyocytes without biomatrix interaction, obtained from signal processing.

FIGS. 13a, 13b, 13c, 13d and 13e illustrate representative voltage maps of neonatal cardiomyocytes interacting with the nanoreinforced bioactive matrix, obtained from signal processing.

### DETAILED DESCRIPTION OF THE INVENTION

This invention originates as a response to the need to solve the different conditions faced by patients suffering from cardiac bioelectric conductivity diseases.

Such condition includes the procedures and the use of medical devices, such as cardiac stimulators, pacemakers and resynchronizers, which bring with them complications associated with the implantation and durability of electronic components.

The description of the embodiment of the present invention is not intended to limit its scope, but to provide a particular example of such invention. The description of this invention allows any knowledgeable person in the subject to understand that the equivalent embodiments stick to the spirit and scope of the present invention in its broadest form.

For a better understanding of the present invention, certain technical terms used in the descriptive chapter will be detailed below.

In the context of the present invention, the term “bioactive implant or patch” means the tridimensional fibrillar network of silk fibroin reinforced with metallic nanoparticles and post-treated with an organic solvent, featuring biocompatible and electroconductive characteristics, validated in an in vitro model of electric stimulus disruption in cardiac cells.

In the context of the present invention, the term “electroconductive polymeric biomatrix” means the tridimensional fibrillar structure of silk fibroin reinforced with metallic nanoparticles and post-treated with an organic solvent, methanol as the preferred modality, featuring electrical and conductive properties.

In the context of the present invention, the term “polar polymer” refers to the polymers having a positive or negative charge, which allows them to generate homogeneous solutions. In addition, a sacrificial polymer refers to a polymer of polar synthetic origin, that will generate fibers to form the electrospun matrix and, thanks to its high hydrophilicity they can be easily removed by dissolving in aqueous media.

In the context of the present invention, the term “electrospun matrix”, refers to the tridimensional structure of silk fibroin, a sacrificial polymer, with or without reinforcement of metallic nanoparticles obtained by means of the electrospinning technique, consisting of a manufacturing process that uses an electric field differential to produce micro- or nanometer-scale fibers.

In the context of the present invention, the term “green synthesis”, refers to the metallic nanoparticle synthesis method that uses biomolecules such as amino acids, enzymes, proteins, polysaccharides, vitamins or fruit or plant extracts as reducer and stabilizer agents, decreasing the risk of releasing toxic residues and enhances biocompatibility in cellular micro-environments.

In the context of the present invention, the term “electromechanical disruption”, refers to the discontinuity of the passage of the bioelectrical stimulus between two areas of the cardiac tissue, which causes a delay or decrease in the propagation of the depolarization wave of cardiac cells.

The present invention refers to the development of an electroconductive polymeric biomatrix of natural origin, from silk proteins, reinforced with metallic nanoparticles, which was validated in a cell-cell and cell-stratum interaction platform, to determine the characteristic of biocompatibility and integration of the biomaterial with the tissue. The bioactive patch intervenes on areas of electromechanical disruption, that is, framed within various tissue block patterns.

In a first object, the present invention corresponds to a bioactive implant or patch, which in the preferred embodiment comprises at least one membrane of an electrospun polymeric membrane of silk fibroin (SF), with a structural reinforcement of gold nanoparticles, which together increase the electroconductive properties of the matrix. Said behavior derives from the ability of nanoparticles and their incorporation into protein fibers to decrease electrical resistance and impedance, and increase electrical conductivity.

In a second object, the present invention discloses a method for manufacturing the bioactive implant or patch, which comprises the stages from the procurement of the silk fibroin material, its interaction with the gold nanoparticles, to its final array in a non-woven fibrillar structure obtained through the electrospinning technique. This manufacturing process uses a differential electric field to produce micro or nanometer-scale fibers, from dissolutions of natural or synthetic-origin polymers. Subsequently, the patch is completely permeated with cell adhesion proteins arranged on the matrix.

The protein used for the development of the bioactive patch of the present invention can be selected from the group consisting of silk fibroin (SF), silk sericin (SS), collagen, fibronectin, elastin, matrigel, albumin, fribin, gelatin, hyaluronic acid, polylysine, polypeptides, polysaccharides (chitin, chitosan), proteoglycans, and combinations and copolymers of the same or equivalent materials known to a person with moderate knowledge about the subject.

To obtain silk fibroin, which is the natural polymer used in the preferred embodiment of the present invention, the source can come from cocoons, silk threads, silk residues or equivalent materials known to a person with moderate knowledge on this subject. Silk may be obtained from worms of the species Bombyx mori, from no-mulberry, mulberry families, Eri, Tasary Muga.

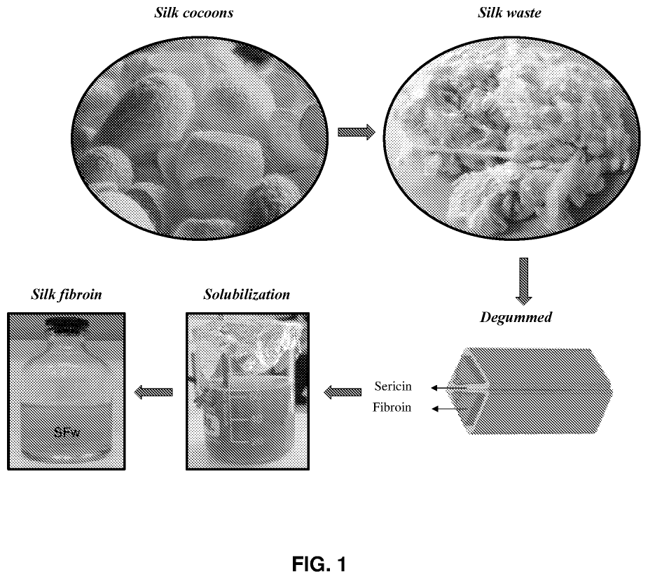

The extraction process may take place by the immersion degumming method, which is performed with any of the following reagents, Na₂CO₃, urea, citric acid, proteolytic enzymes, among others, in an aqueous solution and with continuous stirring. Subsequently, the sample is dissolved in an aqueous LiBr solution with constant stirring. The solution is dialyzed with a cellulose-derived membrane having a pore size between 3500-8000 MWCO, until reaching a stable conductivity; then it is centrifuged at a temperature between 4-8° C., and microfiltered with a membrane having a pore size between 0.8-0.45 μm. Finally, the concentration of waste silk fibroin in aqueous solution is established, as shown in FIG. 1.

In the electrospinning process carried out in the present invention, a sacrificial synthetic polymer is selected, that is, a polymer from the group consisting of polyethylene oxide (PEO), poly (ethylene glycol) (PEG), poly (vinyl alcohol) (PVA), poly (vinyl pyrrolidone) (PVP), polylactic acid (PLA), polyglycolic acid (PGA), polylactic-co-glycolic acid (PLGA), poly (L-Lactide-co-e-caprolactone) or equivalent materials known to a person with moderate knowledge on this subject.

In the preferred embodiment of this invention, the sacrificial synthetic polymer is polyethylene oxide (PEO).

The preferred embodiment of the invention uses green synthesis of gold nanoparticles with a tendency to sphericity, using the concentration of silk fibroin in aqueous solution or silk proteins in a concentration range between 0.5-1% vol/vol as reducing agent and stabilizing matrix, since stabilizing surfactants are not required. In addition, a concentration of the metallic precursor agent, chloroauric acid (HAuCl₄) is used at concentrations between 2.5-3.0 mM; 0.1 N sodium hydroxide (NaOH) and incubation between 32-34° C. to provide stable synthesis conditions. Finally, by means of green reduction, gold nanoparticles (AuNPs-SF) are generated; understanding green reduction as the synthesis process that uses silk fibroin as a reducing agent, and involves the transfer of electrons through oxygen deprotonation, resulting in the formation of a neutral tyrosyl and the reduction of Au⁺³ ions to Au⁰, obtaining metallic nanoparticles as final product.

In a particular embodiment of the invention, the electroconductive polymeric biomatrix has a silk fibroin (SF) in aqueous solution in a range between 4-6% vol/vol, sacrificial polymer such as polyethylene oxide (PEO) in a range between 3-5% m/vol and gold nanoparticles (AuNPs-SF), synthetized by the same silk protein. The final electrospinning solution contains volumetric relations in PEO proportional ranges between 40-60% vol/vol, silk fibroin between 25-50% vol/vol and nanoparticles between 5-45% vol/vol, allowing to obtain a tridimensional fibrillar biomatrix reinforced with metallic nanoparticles.

One of the technical effects of using gold nanoparticles is to increase the electrical properties of the biomatrix. Another of the technical effects of using a polar sacrificial synthetic polymer is to change the viscosity of the solution to be subject to electrospinning in order to promote the stability of the Taylor cone. The Taylor cone is the phenomenon where the drop of solution to go under electrospinning will create an electrostatic repulsion together with the surface tension of the solution itself; this causes the drop to stretch, generating a geometry in the shape of an elongated cone, and the formation of continuous fibers.

Experimental factors were selected from the group that combines the volumetric relationship of (PEO)/silk fibroin (SF)/gold nanoparticles (SF AuNPs) as P50/SF45/Au5%, P50/SF40/Au10%, P50/SF35/Au15%, P50/SF30/Au20%, P50/SF25/Au25%.

In addition, the second experimental factor to be taken into account is the distance and revolutions of the rotary collector of the electrospinning equipment, estimating a distance between 15-25 cm at speeds between 0-250 revolutions per minute (rpm).

The experimental combinations of the biomatrix are comprised in the following list of the PEO/SF/AuNPs-SF volumetric variables, distance and revolutions of the rotary collector of the electrospinning equipment from P50/SF45/Au5% at 15 cm at 50 rpm, P50/SF45/Au5% at 15 cm at 100 rpm, P50/SF45/Au5% at 15 cm at 150 rpm, P50/SF45/Au5% at 15 cm at 200 rpm, P50/SF45/Au5% at 15 cm at 250 rpm. P50/SF40/Au10% at 15 cm at 50 rpm, P50/SF40/Au10% at 15 cm at 100 rpm, P50/SF40/Au10% at 15 cm at 150 rpm, P50/SF40/Au10% at 15 cm at 200 rpm, P50/SF40/Au10% at 15 cm at 250 rpm. P50/SF35/Au15% at 15 cm at 50 rpm, P50/SF35/Au15% at 15 cm at 100 rpm, P50/SF35/Au15% at 15 cm at 150 rpm, P50/SF35/Au15% at 15 cm at 200 rpm, P50/SF35/Au15% at 15 cm at 250 rpm. P50/SF30/Au20% at 15 cm at 50 rpm, P50/SF30/Au20% at 15 cm at 100 rpm, P50/SF30/Au20% at 15 cm at 150 rpm, P50/SF30/Au20% at 15 cm at 200 rpm, P50/SF30/Au20% at 15 cm at 250 rpm. P50/SF25/Au25% at 15 cm at 50 rpm, P50/SF25/Au25% at 15 cm at 100 rpm, P50/SF25/Au25% at 15 cm at 150 rpm, P50/SF25/Au25% at 15 cm at 200 rpm, P50/SF25/Au25% at 15 cm at 250 rpm. P50/SF45/Au5% at 20 cm at 50 rpm, P50/SF45/Au5% at 20 cm at 100 rpm, P50/SF45/Au5% at 20 cm at 150 rpm, P50/SF45/Au5% at 20 cm at 200 rpm, P50/SF45/Au5% at 20 cm at 250 rpm. P50/SF40/Au10% at 20 cm at 50 rpm, P50/SF40/Au10% at 20 cm at 100 rpm, P50/SF40/Au10% at 20 cm at 150 rpm, P50/SF40/Au10% at 20 cm at 200 rpm, P50/SF40/Au10% at 20 cm at 250 rpm. P50/SF35/Au15% at 20 cm at 50 rpm, P50/SF35/Au15% at 20 cm at 100 rpm, P50/SF35/Au15% at 20 cm at 150 rpm, P50/SF35/Au15% at 20 cm at 200 rpm, P50/SF35/Au15% at 20 cm at 250 rpm. P50/SF30/Au20% at 20 cm at 50 rpm, P50/SF30/Au20% at 20 cm at 100 rpm, P50/SF30/Au20% at 20 cm at 150 rpm, P50/SF30/Au20% at 20 cm at 200 rpm, P50/SF30/Au20% at 20 cm at 250 rpm. P50/SF25/Au25% at 20 cm at 50 rpm, P50/SF25/Au25% at 20 cm at 100 rpm, P50/SF25/Au25% at 20 cm at 150 rpm, P50/SF25/Au25% at 20 cm at 200 rpm and P50/SF25/Au25% at 20 cm at 250 rpm.

On the other hand, the relative humidity of the work environment in the electrospinning equipment to obtain the biomatrix is within a range from 25% to 26%, from 26% to 27%, from 27% to 28%, from 28% to 29% and from 29% to 30%.

In addition, the voltage applied to generate the magnetic field to the electrospinning equipment for the present invention, is within a range from 15 kV to 15.1 kV, from 15.1 kV to 15.2 kV, from 15.2 kV to 15.3 kV, from 15.3 kV to 15.4 kV, from 15.4 kV to 15.5 kV, from 15.5 kV to 15.6 kV, from 15.6 kV to 15.7 kV, from 15.7 kV to 15.8 kV, from 15.8 kV to 15.9 kV and from 15.9 kV to 16.0 kV.

In addition, in order to obtain a matrix, an electrospinning equipment is used, having a 21 G glass syringe that contains a PEO/SF/AuNPS solution.

In order to obtain a continuous non-woven textile in the rotary collector, a flow between 0.3-0.9 ml/h is generated, using a syringe injection pump, a high voltage source to create a positive and negative flow, and aluminum foil coated metal tubes are used with ground connection as collector substrates.

In the present invention, one or several organic solvents are selected to remove the sacrificial synthetic polymer, from the group consisting of methanol, ethanol, propanol, butanol, glutaraldehyde (GA), acetone or equivalent materials known to any person with moderate knowledge on this subject.

In order to understand the present invention, the post-treatment process with organic solvents will be understood as the procedure for removing the sacrificial synthetic polymer (in the preferred modality, polyethylene oxide PEO) from the three-dimensional structure obtained from the electrospinning process. One of the technical effects is to promote the transition of the silk fibroin macromolecules and their non-crystalline secondary structures to crystalline structures in the form of sheets and β turns, increasing the crystalline phase of the membranes and making them insoluble in water. Finally, washing is conducted with deionized water at 37° C. One of the technical effects of using this process is to remove traces of the synthetic polymer present in each one of the biomatrices.

For the characterization process of the biomaterial, the characterization pathway of the physical-chemical and electrochemical properties is used. The outcome is that the biomatrix has a fibrillar morphology with intertwined fibers created by a non-woven texture, the presence of gold nanoparticles arranged in the fibers of the biomatrix, changes in the functional groups present in the membranes, topographic profiles with potential differentials of the surface of the nanoparticle reinforced electrospun biomatrix, and impedance, resistance, and electrical conductivity assays.

The foregoing allows to determine that the biomatrix has a fibrillar structure with uniform fiber diameters at nanometric scale. It is also observed that the nanoparticles are embedded in all the fibers of the biomatrix. Likewise, the biomatrix displays an increased electrical conductivity and less opposition to current flow.

A post-treated gold nanoparticle-reinforced fibroin membrane is used as a bioactive biomatrix for biocompatible and functional evaluations in a pattern of electrical impulse disruption in cardiac cells.

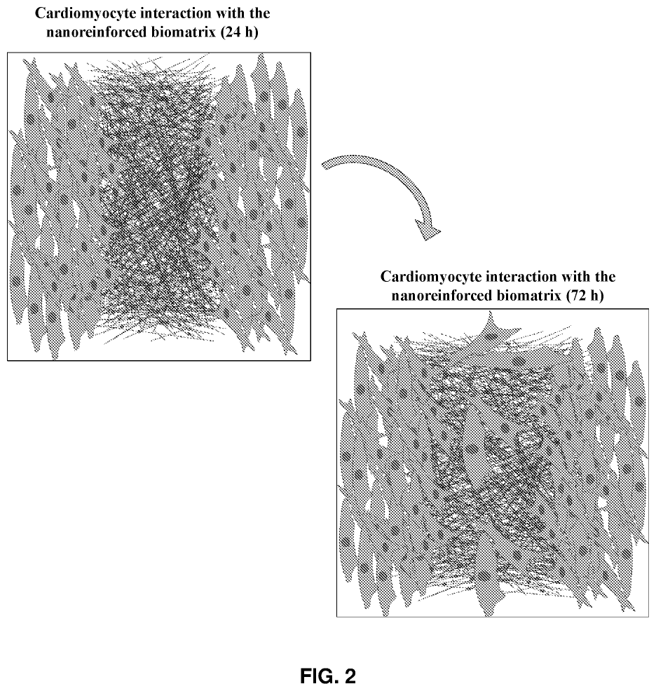

FIG. 2 illustrates cell patterns with linear architecture emulating the cardiac cell disruptions, promoting a cardiomyocyte biomatrix interaction pattern. It is then found, that the cells coexisted and migrated to the membrane creating cell syncytia with synchronous electrophysiological response of cardiomyocytes, a behavior that is promoted with the incorporation of gold nanoparticles embedded in the fibrillar structure, made from natural origin proteins such as silk fibroin.

The following are intended to describe the preferred aspects of the invention, however, such examples do not pretend to limit its scope.

### Example 1

Silk fibroin was obtained from agricultural residues in aqueous solution at a concentration between 4.5-5% vol/vol, and stored at 4-5° C. Afterwards, a PEO solution between 3.5-4.5% m/vol, was made, using distilled water as a solvent for the synthetic polymer, which was continuously stirred for 48 hours until obtaining a homogeneous solution. On the other hand, a solution of chloroauric acid (HAuCl₄) was prepared, at a concentration between 2-2.5 mM and stored at a temperature between 4-8° C. In addition, 09.1 N Sodium hydroxide was prepared.

In order to obtain gold nanoparticles from the green synthesis using silk fibroin, a silk fibroin solution was prepared, at a concentration between 0.3-0.6% vol/vol and was mixed with a solution of chloroauric acid (HAuCl₄) at a concentration between 2-2.5 mM. Subsequently, the pH was adjusted to 9-10 with 0.1 N sodium hydroxide.

Next, it was incubated under white light for 20-24 h at 32-34° C. Finally, the nanoparticle solutions were stored and protected from light at 4-8° C.

A mix of silk fibroin at a concentration of 4-5% vol/vol, polyethylene oxide (PEO) 3.5-4.5% m/vol and gold nanoparticles was prepared. The mixture was stirred at low revolutions for 15 to 30 min in order to homogenize the solution to be electrospinned. The mixture was deposited in a 21 G glass syringe and was placed in a single channel pump at a flow of 0.5-0.8 ml/h, voltage between 15-16 kV, at a distance from the syringe needle to the collector between 18-20 cm, rotation speed 200-250 rpm, relative humidity 26-30% and was subject to electrospinning for 3-6 h. An aluminum coated stainless steel rod was used as collector.

After the electrospinning, the collector is removed and the aluminum foil sample is dismantled. The biomatrix was placed in a Petri dish where the 90% methanol pre-treatment solution was placed until the biomatrix was immersed for 10-15 min. Next, the solvent was removed and was put in a controlled vacuum atmosphere for 20-24 h. Finally, the biomatrix was washed with deionized water at 37° C., for 45-48 h.

As control biomatrix, a silk fibroin solution was electrospun at a concentration between 4-5% vol/vol and polyethiylene oxide at a concentration between 3.5-4.5% m/vol, flow between 0.5-0.8 ml/h, voltage 15-16 kV, distance from the syringe needle to the collector of 18-20 cm, rotatory speed 200-250 rpm, relative humidity 26-30% and then was subject to electrospinning for 3-6 h. An aluminum coated stainless steel rod was used as collector. This control biomatrix was also post-treated in accordance with the protocol described for the gold nanoparticle reinforced biomatrix.

To obtain the biomatrix, polyethylene oxide was used as sacrificial polymer, which was removed from the final membrane through post-treatment processes with organic solvents selected from the following group: methanol, ethanol, propanol, glutaraldehyde (GA), in the preferred modality with methanol. Subsequently, the electroconductive polymeric biomatrix is characterized using scanning electronic microscopy (SEM) and field scanning transmission, Fourier transform infrared spectroscopy, visible ultraviolet spectrometry, electrochemical impedance spectroscopy and atomic force microscopy.

An in vitro model of primary heart cells was used as a biological validation method. These cells were extracted from 1-3 days old neonatal mice, using the cold enzymatic digestion methodology with type I collagenase and trypsin. Subsequently, a blocking pattern of the native bioelectrical stimulus of cardiac cells was generated, emulating the conduction tissue blocks of the excito-conductive system.

Once the biomatrix is obtained, it is arranged in an in vitro model that emulates the conditions of bioelectric conduction disruption of cardiac cells. Then, the characteristics of biocompatibility and electrophysiological functionality are determined by tests for cell adhesion, proliferation and structure changes. Subsequently, intracellular and extracellular calcium flow signals, membrane potential and voltage maps of the conduction velocity of the biological model in interaction with the biomatrix were obtained.

The characterization is used to determine that the biomatrix shows a fibrillar structure at nanometric scale, which contains embodied gold nanoparticles throughout its tridimensional network, that confers the ability to modulate charge transfer and modify the conduction speed of the electric stimulus. In addition, the fibrillar network framework of the biomatrix with gold nanoparticles presents a beneficial topographic signal for cardiac tissue engineering.

On the other hand, the electroconductive biomatrix reinforced with gold nanoparticles supports the action provoked by the intracellular calcium homeostasis due to the low resistivity and impedance of the biomaterial. In addition, there is an increase of the amplitude of biosignals and the heartbeat frequency of cardia cells in interaction with the biomatrix, indicating that this type of biomaterial can be considered a therapeutic strategy for problems associated to electrical conduction pathways.

### Example 2. Characterization

### UV-Visible Spectrometry and Electronic Microscopy of Field Scanning Transmission

In order to determine the formation and presence of gold nanoparticles in green synthesis and in the electrospun biomatrices, the spectrophotometer was operated in a range of wavelengths between 200-1100 nm. On the other hand, the morphologic characteristics of the fibrillar structure and the size of the gold nanoparticles were analyzed in a field emission scanning electron microscope in STEM Nova NanoSEM 200 mode operated at 15 kV.

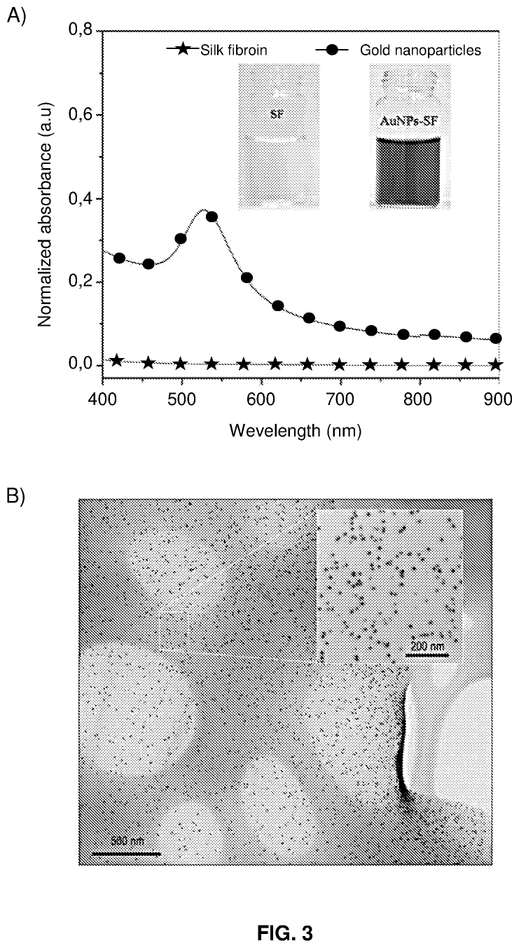

FIG. 3a shows absorption spectra corresponding to the surface plasmon resonance (SPR) for the gold metal ion and its silk fibroin control. It is also evident the presence of spectral bands at wavelengths between 522-528 nm, which are typical resonance bands for gold nanoparticles. The color change of the solutions from a pale white to a red, indicating the formation of gold nanoparticles was visualized.

FIG. 3b shows micrographs and histograms of gold nanoparticles. It is evident that the gold nanoparticles have a tendency to sphericity with an average particle diameter of 12 nm, and a standard deviation of 3 nm.

On the other hand, the polydispersity index (PDI) of the gold nanoparticles was obtained. The (PDI) value was 0,048, which is a dimensionless value used to describe the degree of dispersion of the particle size distributions. Therefore, it was determined that the particle diameters are mono-dispersed with a uniform size particle.

With respect to the colloidal stability provided by the silk fibroin to the gold nanoparticles through the presence of the groups, semiquinone, amine and carboxylic acid in the protein peptid chain, an electrokinetic potential analysis was conducted, finding a value of −40.2 mV for the gold nanoparticles. It was determined that the nanoparticles have a negative charge and are stable, and this behavior is due to their monodispersibility which causes the electrostatic repulsion voltage between the particles to be stronger than the Brownian random thermal motion.

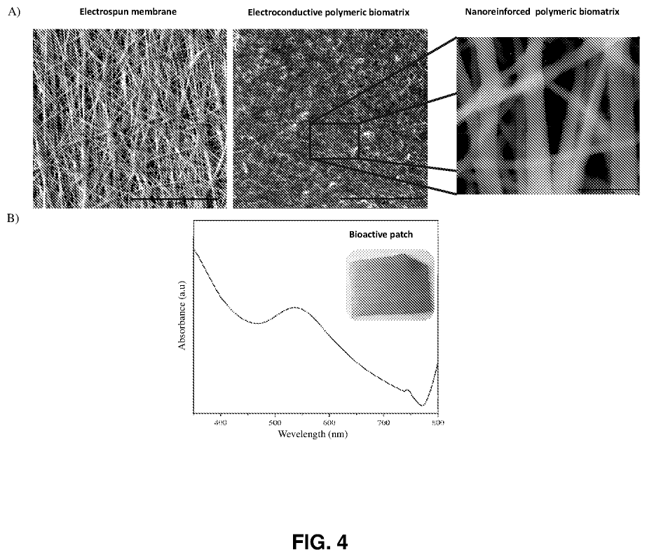

FIG. 4a shows the tridimensional network morphology of the electrospun matrices of silk fibroin, polyethylene oxide and gold nanoparticles, evidencing a fiber diameter of 79 nm and a standard deviation of 25 nm. After the post-treatment process, the fibers became thicker and the pore diameter was reduced. This behavior was the result of removing the polyethylene oxide from the electroconductive polymeric biomatrix. In addition, the qualitative analysis showed that the gold nanoparticles are dispersed in the fibers and no lumps were formed. On the other hand, the electroconductive polymeric biomatrix, evaluated by UV Vis, showed absorption spectra between 500 and 600 nm corresponding to the surface plasmon resonance for the gold ion. In addition, the color of the biomatrix turned pink, a characteristic color of the gold nanoparticle solution used to develop the material (FIG. 4b).

### Fourier Transform Infrared Spectroscopy

With respect to the changes in the functional groups present in the biomatrixes, a Nicolet iS50 spectrum with an attenuated total reflectance (ATR) module was used at a resolution of 4 cm⁻¹ and 32 scans. The infrared spectra obtained were used to determine the crystallinity percentage of biomatrices through the deconvolution of the peak corresponding to the amide vibration 1 (1600-1700 cm⁻¹).

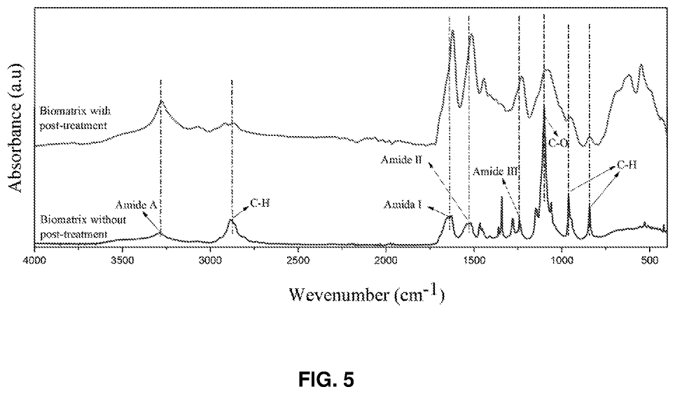

FIG. 5 shows the FTIR spectra of the chemical changes that conform the functional groups of the electrospun biomatrices with nanoparticles, after post-treatment with methanol. The spectra of the electrospun biomatrix reinforced with metallic nanoparticles without post-treatment process shows the presence of absorption bands at wavelengths close to 2877 cm⁻¹ (aliphatic C—H stretches), 1098 cm⁻¹ (C—O stretches), 961 cm⁻¹ (C—H out of the plane) and 841 cm⁻¹ (C—H out of the plane), which correspond to the presence of PEO in the biomatrix fibrillar network. On the other hand, there were absorption bands at 3286 cm⁻¹, 1638 cm⁻¹, 1528 cm^{-1 and} 1241 cm⁻¹, characteristic peaks of the amide A (N—H stretch), amide I (C═O stretch), amide II (N—H flexion and de C—N stretch) and amide III (C—C, C—N stretches and C—H flexion), respectively.

When comparing the FTIR spectra of the electrospun matrix with the electroconductive polymeric biomatrix, it was found that, in the case of the electroconductive biomatrix, the amide I and II bands shifted to 1619 cm⁻¹ y 1509 cm⁻¹. This behavior is the result of the transition from helix structures α or random spiral structures to β-sheet or β-turn conformation. By washing the electroconductive biomatrix for 48 h at 37° C., it is possible to extract the PEO, which was confirmed by the absence of the characteristic absorption peaks in the FTIR spectra.

From the FTIR spectra, the percentage of the crystalline structures contained in the amide I band was determined. It was found that the electrospun matrix without post-treatment showed 39.3% of p sheets, 0% of p turns, 0.4% of lateral chains, 12.1% random spirals, 42.7% α-helixes and 5.5% turns. These percentages changed in the biomatrix post-treated with methanol, with the following results: 50.5% p sheets, 44.2% p turns, 5.4% lateral chains and 0% random spirals, α-helixes and turns. Therefore, this confirms that when using alcohols in the treatments, in this case methanol, the crystallinity of the biomatrix increased due to the transition from macromolecules and the non-crystalline secondary structures (α-helixes, random spirals or turns) to crystalline structures (β sheets or β turns), making the nanoreinforced polymeric biomatrix to be insoluble on contact with aqueous solutions.

### Electrochemical Impedance Spectroscopy

The nanoparticle reinforced electrospun biomatrix and the post-treated control biomatrix were evaluated by impedance electrochemical spectroscopy (IES) in order to determine their electroconductive properties. The configuration consisted of an array of three electrodes, where the biomatrix was arranged over a graphite rod performing as a work electrode (WE) with an approximate exposed area of 8 cm². Likewise, a graphite rod and an Ag/AgCl electrode were used as counter-electrode (CE) and reference electrode (RE), respectively. A Hanks's balanced saline solution without Ca²⁺ y Mg²⁺ (HBSS) was used as electrophysiological solution. The recording of the measurements to determine the impedance, resistance and electrical conductivity was carried out in an Ivium CompactStat potentiometer with scanning frequency from Hz at 500 kHz with 10 point per decade and 160 mV of amplitude of the AC potential.

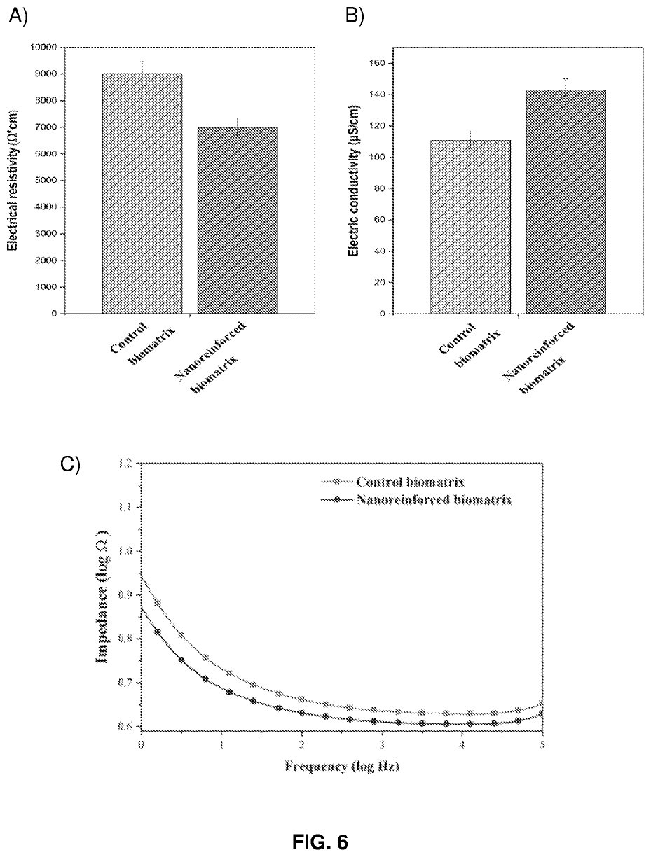

FIG. 6a shows the electrical resistivity of the biomatrix reinforced with gold nanoparticles and the control biomatrix without nanoparticles after the post-treatment process, that reported a resistivity of 6995 and 9017 Ω*cm, respectively. Based on the results obtained from the biomatrices, it was determined that when including gold nanoparticles in the fibrillar structure, a lower resistance occurred when electrons were flowing, compared to the unreinforced biomatrix. This behavior is due to the greater alignment and uniform distribution of fibers in the structural framework of the biopatch.

Furthermore, FIG. 6b shows the electrical conductivity of the developed biomatrix and the control biomatrix both post-treated, with values of 143±7 μS/cm y 111±5 μS/cm, respectively. It was observed that the biomatrix reinforced with gold nanoparticles has a higher electrical conductivity, that is, the biomaterial has the ability to conduct electrical current through it. The analysis of the results obtained from the nanoreinforced biomatrix show that when including gold nanoparticles in the atomic and molecular structure of the biomatrix, an increase of the conductivity occurs. This behavior is due to a high number of electrons with weak interactions, which facilitates the motion of such electros in the fibrillar structure of the biomatrix.

On the other hand, FIG. 6c shows the influence of physical and chemical phenomena of biomatrices when applying a voltage of 160 mV. The EIS records evidenced that the biomatrix and its control, showed that at high frequencies (100000-1000 Hz) the impedance magnitude was 0,609 and 0.635Ω, respectively, and that at low frequencies (1000-0.1 Hz) the impedance increased to 0,871 and 0.943Ω, respectively. From the diagrams, it can be inferred that the biomatrix reinforced with gold nanoparticles has a lower impedance magnitude at high and low frequencies, compared to the non-reinforced control biomatrix. This behavior is due to the type of particle that reinforced the biomatrix, which causes a greater displacement of electrons in the fibers of the tridimensional structure.

### Kelvin Probe Force Microscopy

To determine the topography and power differential of the nanoparticle biomatrix surface, an Asylum Research atomic force microscope and a Ti/Ir (5/20) silicone coated tip with a radio of 28±10 nm was used. Several graphs of distance versus voltage were made to determine the contact potential differential.

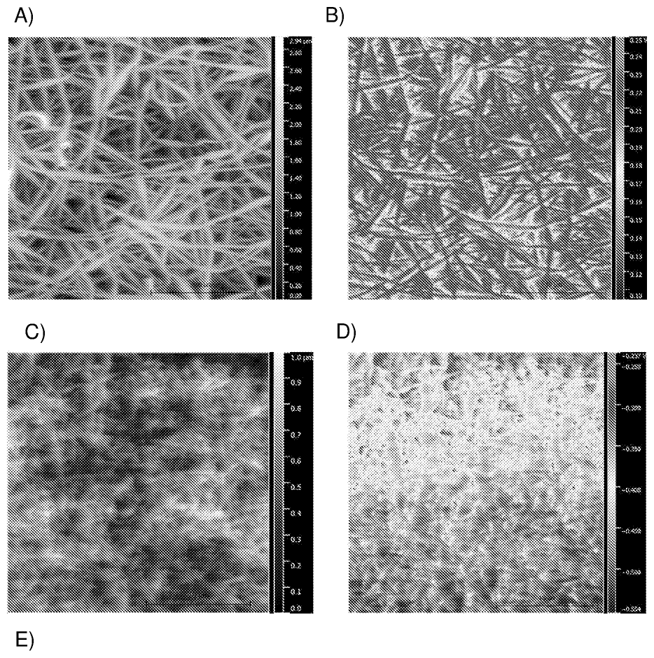

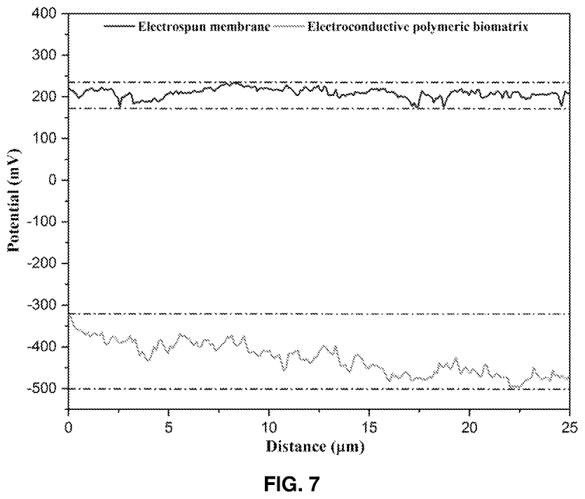

FIG. 7a shows the topography of the reinforced biomatrix without post-treatment, revealing homogeneous fibers with no defects. Also, FIG. 7b shows the contact potential difference with values between 0.10 V and 0.25 V. After the post-treatment process of the biomatrix reinforced with gold nanoparticles, it was found that a homogeneous fibrillar morphology (FIG. 7c) is preserved, and when comparing the height of the topography with the electrospun biomatrix without post-treatment, the height decreased from 2.94 μm to 1 μm.

This decrease is due to the extraction of the synthetic polymer (PEO) from the biomatrix, which was present in 50% of its final structure. On the other hand, the surface potential differential of the methanol-treated biomatrix went from presenting a positive potential to a negative potential with values between −0.237 V and −0.554 V (FIG. 7d) when compared to the electrospun matrix without post-treatment. For this reason, the change in the surface potential difference correlates directly with the change in the surface chemistry of the nanoreinforced electrospun biomatrix, where the post-treatment with organic solvents exposes negatively charged amino acids, such as aspartic acid and the glutamic acid on the surface.

FIG. 7e shows the behavior of the electrical potential (mV) versus the position (μm) of the gold nanoparticle reinforced biomatrix with and without post-treatment. The upper part of the graph shows the variation of the surface potential with minimum values of 0.18 V and a maximum peak of 0.22 V for the untreated electrospun matrix and a difference between the two of 61 mV, while the lower part of the graph shows the variation of the surface potential of the post-treated biomatrix, showing as minimum values −0.66 V and maximum values −0.52 V, and a difference between the two of −0.13 V.

Based on the results it can be inferred that the signals are modified as a result of the electrostatic interactions on an atomic scale and short range forces caused by the generation of charged ionic species, that could conduct an electric stimulus as a result of the change in the polarity of the biomatrix.

### Immunofluorescence Study of Structural Cellular Changes

Fluorescent microscopy was used to determine the interaction between the nanoreinforced biomatrix and primary cardiac cells. Monoclonal antibodies were directed towards the following proteins: Hoechst (nucleus), a (actin protein binding) and conexin 43 (intercellular communication). The biomatrix was sterilized by ultraviolet radiation for 30 minutes on both faces of the fibrillar structure.

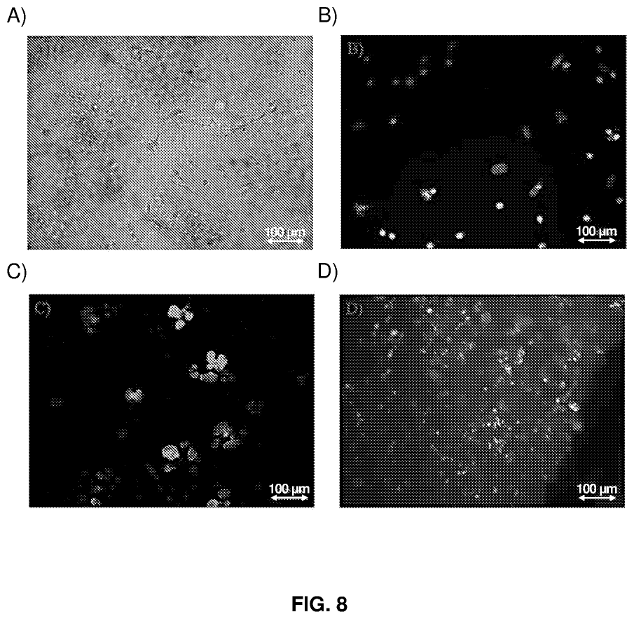

FIG. 8a shows bright field images of the cell interaction of the primary cardiomyocytes with the gold nanoparticle reinforced biomatrix during an incubation period of 24 hours, where the coexistence of cells interacting with the biomatrix and the generation of communicating bonds on its surface, as the incubation period increased was observed. This indicated that this type of biomatrix does not cause disaggregation nor loss of cell interaction.

The immunofluorescence analysis based on the identification of nuclei and a actin showed that the gold nanoparticle reinforced biomatrix supports the generation of cellular syncytia, and also enables the expression of actin anchoring points. This protein participates in establishing the network of microfilaments and together with the catenins and cadherins is involved in the cell-cell communication processes supporting cell adhesion (FIGS. 8b and 8c).

Connexin 43 (Cx43) coupled to green fluorescent protein was used as a marker to identify the gap junctions. The expression Cx43 is important to determine the cell-cell communication processes and to ensure the homeostasis and transfer of biological information between neighboring cells after an incubation period of 72 h (FIG. 8d).

Based on the results of the immunofluorescence, it was determined that the gold nanoparticle reinforced and post-treated biomatrix enables the expression of cell communication structures and contractibility, which are important characteristics to determine the functionality of this type of biomaterials in capturing the electrical stimulus and the propagation of the action potential from one area to another of the cardiac tissue.

### Identification of Calcium and Membrane Potential by Optical Mapping

An optical mapping (OM) fluorescent system using fluorescent probes was implemented to measure the calcium flow and the plasma membrane potential in cardiomycytes cell cultures, subject to cell block patterns in interaction with the post-treated nanoreinforced biomatrix. The OM system comprised a light source, a high-time resolution, high quantum efficiency and high signal-to-noise ratio photodetector. The calcium measurements used an Indo-1 AM dye, a radiometric probe that excites at 346 nm and renders a signal at 475 nm. In the case of the membrane potential, the Dibac₄ (3) dye was used, a probe sensible to low response potential that is excited at 490 nm and renders a signal at 516 nm.

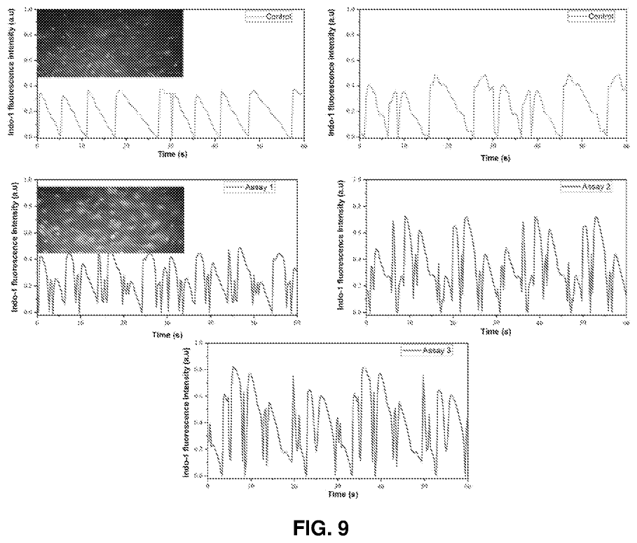

FIG. 9a shows a representative image of the calcium response in neonatal cardiomyocyte cultures with no intervention with the biomatrix, observing variations in the fluorescence provoked by the action potential of the cells, which triggered the activation of the voltage-dependent L-type calcium channels and also the release of the intracellular calcium reservoir from the sarcoplasmic reticulum, which activates the contraction.

On the other hand, it was determined that the cardiomyocytes when interacting with the gold nanoparticle reinforced biomatrix, showed variations in the intensity of the fluorescence associated with the intracellular calcium movement, which is related to the excitation and contraction of the heart cells. (FIG. 9b). In this case, there was an increase of the intensity of the calcium signal in cardiomyocytes interacting with the nanoreinforced biomatrix compared to cells without biomatrix, finding peaks of calcium intensity of the treated cells between 0.4 and 0.8 compared to cells alone ranging between 0.3 and 0.4. In addition, in the cardiomyocytes cultures with the biomatrix, an increase in the frequency of the calcium wave was observed, a rapid entry of the calcium into the cytosol and a rapid exit of this ion, favoring the process of cell repolarization compared with the cardiomyocytes cultures without the biomatrix, that reported slow and prolonged calcium releases, as well as a lower fluorescence amplitude intensity (FIG. 9b).

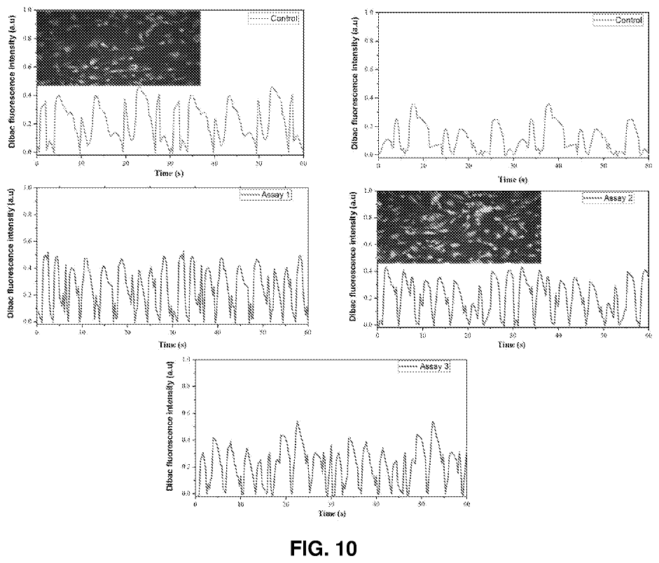

FIG. 10 shows a representative image of the fluorescence emission associated with the plasmatic membrane potential of the cardiomyocytes with and without interaction the nanoreinforced biomatrix (FIG. 10a), where an increase in cell depolarization is related to the additional inflow of the potentiometric probe, which is translated into an increase of fluorescence. From the above, fluorescence was present in cardiomyocyte cultures, but the cells interacting with the gold nanoparticle reinforced biomatrix showed a more homogenous potential intensity compared to cells without biomatrix, indicating that this tridimensional structure increases cell depolarization.

Voltage changes were determined from the fluorescence records of the membrane potential of the cardiomyocyte culture. Such voltages represent the ratio of fluorescence intensity of the cell membrane divided by the background intensity. Furthermore, FIG. 10a shows typical signals of depolarization and repolarization of the plasmatic membrane of cardiac cells with a frequency of 8 wave complexes in a period of 60 s; on the other hand, the intensity signals associated with cardiomyocytes interacting with the nanoreinforced biomatrix increased the fluorescence amplitude and depolarization frequency (FIG. 10b), finding 15 and 18 depolarization and repolarization wave complexes in the same assessment interval. Consequently, it was determined that cardioymyocites interacting with the biomatrix modulate the action potential and generate a higher heartbeat frequency, which could explain the electro-conductive property of this newly developed biopatch.

### Cardiac Electrophysiological Signals by Microelectrode Array

A microelectrode array platform (MEA) that comprises an amplifier, a data acquisition software, a stimulator, a temperature controller and microelectrode matrixes were used. In order to obtain the bioelectrical signals of the cardiomyocyte cell culture interacting with the gold nanoparticle reinforced biomatrix, a pattern to emulate in vitro the block action of the cardiac tissue was used. In addition, an algorithm was implemented to obtain the extracellular field voltage activation maps that showed the signal propagation pathways. Measurements were made on cell cultures using microelectrodes in unipolar configurations at 37° C., for 10 s at a sampling rate of 10 kHz.

FIG. 11a, shows the signals of 4 cardiomyocyte recording channels without interaction with the biomatrix, evidencing a beat frequency per minute of 98±1. This behavior is due to the presence of electrical potentials resulting from the change in polarity of the plasma membrane due to the activation of the ion channels that produce a flow of calcium sodium and potassium at intra-cellular level. Regarding the bioelectrical response of the cardiomyocytes in interaction with the nanoreinforced biomatrix, the heartbeat frequency increased to 123±1 beats per minute (FIG. 11b).

In the same way, voltage activation maps are made to visualize the cellular depolarization and the behavior of the conduction speed of cardiomyocyte syncytia. A color range was used to read the voltage maps, indicating the moment when the action potential begins and propagates. In this case, red represents the maximum intensity of the extra-cellular field potential, and blue the cell resting potential.

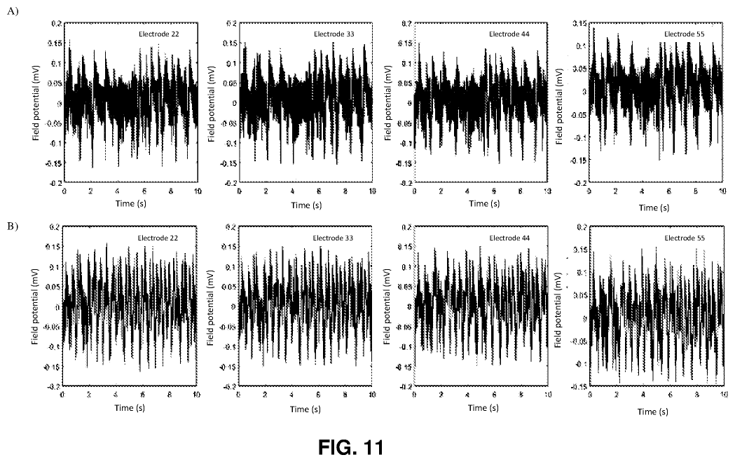

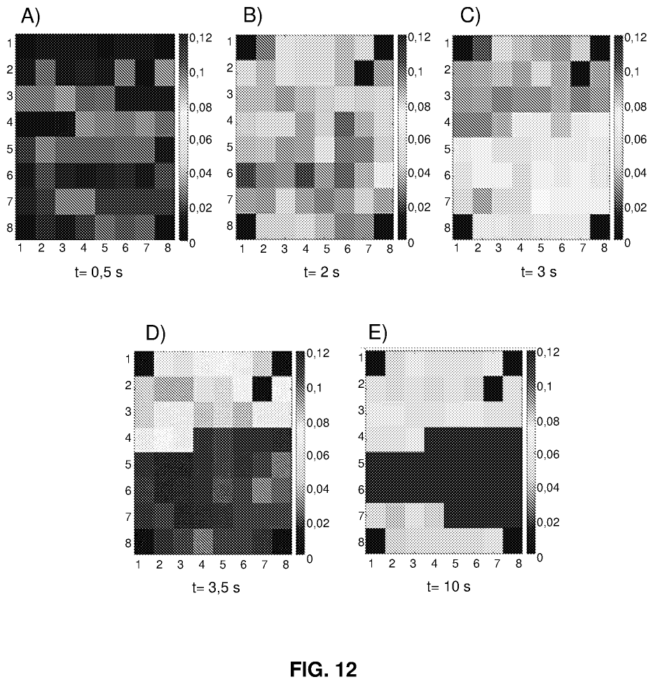

FIG. 12a shows the voltage maps representing the behavior of cariomyocyte electrical potential without interacting with the biomatrix, which show that at an experimental time of 0.5 s there is no action potential, and as the registry time increased, after 2 s the cardiomyocytes depolarized, which was evidenced by the change in color intensity of the events captured in the positions of the electrodes located in the lower part of the voltage map (FIG. 12b). Likewise, an almost total propagation of the extra-cellular action potentials occurred at 3.5 s (FIG. 12d). This behavior is due to the non-synchronic depolarization of cardiomyocytes due to the spontaneous expression of the action potentials.

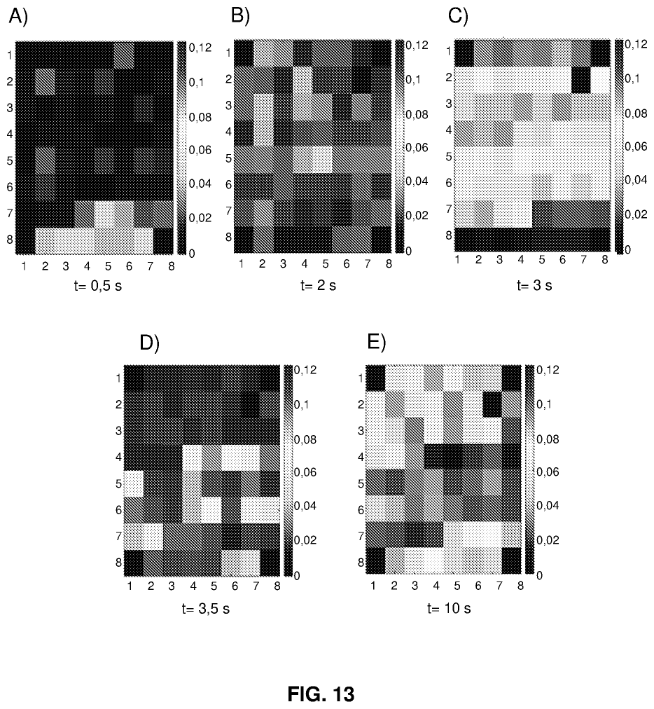

By contrast, FIG. 13a shows voltage maps representing the bioelectric activity of cardiomyocytes upon interaction with the electroconductive polymeric biomatrix, showing an activation and uniform propagation of action potentials. At 0.5 s of experimental recording, there was an activation of the electrodes located between position 7.5 and 8.7 of the microelectrode array matrix, indicating an earlier onset of cardiomyocyte depolarization. Likewise, as the recording time increased, propagation of the action potential of the cardiac cell syncytia occurred, and a higher frequency of the electrical activity of the cells compared to the voltage maps of the cells without biomatrix. At 2 and 3.5 s a uniform intensity change in the red range was evidenced in the voltage maps (FIGS. 13b y 13d). Said behavior is homologous with the biosignals presented in FIG. 11b, that showed an increase in the beat frequency.

The fibrillar microarchitecture of the electroconductive polymeric biomatrix of aqueous solutions of the silk fibroin and reinforced with gold nanoparticles, synthetized from the same protein, showed an increased electrophysiological behavior of cell syncytes of native cardiomyocytes. In addition, there was an increase of the expression of calcium ions supporting the excitation and contraction of cardiomyocytes, that was evidenced with an increase in the heartbeat frequency and the propagation of the action potentials visualized in the voltage maps.

It should be understood that the present invention of the implant or cardiac electroconductive nanoreinforced polymeric biomatrix is not limited to the modalities described and illustrated herein. As it is evident for a person with knowledge of this technical subject, there are multiple variations and modifications, that when preserving the spirit and scope of this invention, are within the scope of the annexed claims.

1. A bioactive implant for restoring the conductivity of the bioelectrical stimuli in cardiac tissue comprising polymeric matrix of silk fibroin in aqueous solution previously electrospun with a polar sacrificial polymer, reinforced with a gold nanoparticle membrane synthetized with silk fibroin.

2. The bioactive implant in accordance with claim 1, characterized because the polar sacrificial polymer is selected from the group consisting of polyethylene oxide (PEO), poly (ethylenglicol) (PEG), poly (vinylic alcohol) (PVA), poly (vinyl pyrrolidone) (PVP), polylactic acid (PLA), polyglycolic acid (PGA), polylactico-co-glycolic acid (PLGA), poly (L-lactid-co-ε-caprolactone).

3. The bioactive implant in accordance with claim 1, characterized because the polar sacrificial polymer and the silk fibroin are in a ratio between 45:50 up to 25:50% vol/vol.

4. The bioactive implant in accordance with claim 1, characterized because the gold nanoparticles have a particle diameter between 5 and 19 nm.

5. The bioactive implant in accordance with claim 1, characterized because it has a fiber diameter between 35 and 150 nm.

6. The bioactive implant in accordance with claim 1, characterized because it has a tridimensional structure.

7. The bioactive implant in accordance with claim 1, characterized because the reinforced membrane of gold nanoparticles synthetized with silk has a concentration from 5 to 25% vol/vol with respect to the final solution.

8. A method for manufacturing a bioactive implant for restoring the conductivity of the bioelectrical stimuli in cardiac tissue comprising the following stages:
Provide silk fibroin in aqueous solution at a concentration between 4.5-5% vol/vol;
synthetize gold nanoparticles in a silk fibroin aqueous solution at a concentration between 0.3-0.6% vol/vol;
mix the silk fibroin solution with a polar sacrificial polymer selected from the group consisting of polyethylene oxide (PEO), poly (ethylene glycol) (PEG), poly (vinyl alcohol) (PVA), poly (vinyl pyrrolidone (PVP), polylactic acid (PLA), polyglycolic acid (PGA), polylactic-co-glycolic acid (PLGA), poly (L-lactide-co-e-caprolactone), where the polar sacrificial polymer is in a concentration between 3.5-4.5% m/vol;
mix the silk fibroin solution and the polar sacrificial polymer with the synthetized gold nanoparticle solution;
the mix obtained is subject to electrospinning through sequential rotary electrospinning;
immerse the electrospun matrix in a solution of organic solvent selected from the methanol, ethanol, propanol, butanol, glutaraldehide (GA), acetone group;
place in a controlled vaccum atmosphere for 20-24 h;
wash with deionized water;
obtain the bioactive implant.

9. The method to manufacture the bioactive implant of claim 7, characterized because in the synthesis stage of gold nanoparticles, the silk fibroin solution is mixed with a chloroauric solution (HAuCl₄) at a concentration between 2-2.5 mM.

10. The method to manufacture the bioactive implant of claim 7, characterized because the synthesis stage of the gold nanoparticles includes a pH adjustment stage to a value between 9-10 with 0.1 N sodium dioxide and incubation under white light for 20-24 h at 32-34° C.

11. The method to manufacture the bioactive implant of claim 7, characterized because the electrospinning is carried out at a rotor speed from 50 to 250 rpm, and a needle-collector distance between 15 and 20 cm.

12. The method to manufacture a bioactive implant of claim 7, characterized because in the washing stage, the deionized water is at 37° C., for 45-48 h.
